(19) United States
(12) Patent Application Publication
(10) Pub. No.: US 2024/0261225 A1
(43) Pub. Date: Aug. 8, 2024

(54) FORMIC ACID AS PROCESSING AID IN SPRAY DRYING FOR BASIC DRUGS

(71) Applicant: Lonza Bend Inc., Bend, OR (US)

(72) Inventors: Michael Morgen, Bend, OR (US); Warren Miller, Bend, OR (US); David Vodak, Bend, OR (US); Jonathan Cape, Bend, OR (US); Molly Adam, Bend, OR (US); Michael Grass, Bend, OR (US)

(73) Assignee: Lonza Bend Inc., Bend, OR (US)

(21) Appl. No.: 18/565,885

(22) PCT Filed: Jun. 3, 2022
(86) PCT No.: PCT/EP2022/065249

§ 371 Date: Nov. 30, 2023

Related U.S. Application Data
(60) Provisional application No. 63/196,761, filed on Jun. 4, 2021.

(30) Foreign Application Priority Data
Jun. 29, 2021 (EP) 21182426.3
Jan. 7, 2022 (EP) 22150583.7

(51) Int. Cl. A61K 9/16; A61K 31/506; A61K 31/5377

(52) U.S. Cl. CPC A61K 9/1682; A61K 9/1617; A61K 9/1652; A61K 31/506; A61K 31/5377

(57) ABSTRACT

The invention discloses a method for preparation of spray dried solid dispersions, SDD, comprising an active agent, AA, such as an active pharmaceutical ingredient, API, and a dispersion polymer, DISPPOL, wherein the spray drying is done with a solution of AA and of DISPPOL in a solvent comprising C₁₋₃ alkanol and formic acid, and optionally water.

The invention discloses a method for preparation of spray dried solid dispersions, SDD, comprising an active agent, AA, such as an active pharmaceutical ingredient, API, and a dispersion polymer, DISPPOL, wherein the spray drying is done with a solution of AA and of DISPPOL in a solvent comprising C₁₋₃ alkanol and formic acid, and optionally water.

### BACKGROUND OF THE INVENTION

Spray dried solid dispersions, SDD, comprising an active pharmaceutical ingredient, API, and a dispersion polymer, DISPPOL, are typically produced by dissolving the dispersion polymer and the API in a volatile solvent, such as methanol or acetone, or in a mixture of solvents, followed by spray drying. In cases where the API has limited solubility, e.g. <4 wt % at room temperature, in the spray drying solvent, an API suspension can be heated to a temperature either below or above the solvent's ambient pressure boiling point, this is known as “hot spray drying process”, resulting in a higher dissolved concentration of API. In some cases, even the higher temperatures do not give adequate API concentrations that are economical for a spray drying process, or cause other problems such as chemical degradation of the API, or bear the risk of incomplete API dissolution in the heat exchanger. Alternate, non-preferred volatile solvents can provide increased solubility of the API, but these solvents have other disadvantages that make them less desirable, e.g. high cost, toxicity, poor equipment compatibility, poor commercial availability, high disposal costs, challenges removing to sufficiently low levels, higher viscosity.

WO 2019/220282 A1 discloses in Example 1 spray drying of a solution of erlotinib and a dispersion polymer (PMMAMA or hydroxypropyl methylcellulose acetate succinate H grade) in methanol to provide a spray dried dispersion.

WO 2017/108605 A1 discloses in table 1 of the examples spray-drying of a mixture containing dasatinib, polyvinylpyrrolidone (PVP) and a solvent containing ethanol:aqueous HCl (3M):and water 60:5:35.

US 2014/343073 A1 discloses in example 25 spray-drying of a mixture containing dasatinib, HPMC-AS, citric acid hydrate and a solvent containing ethanol and water.

WO 2014/081581 A2 discloses in example 1 spray-drying of a solution containing efavirenz, a polymer such as HPMCAS, acetone and methanol.

WO 2011/082426 A1 discloses in example 6 the preparation of beads by spraying of a solution of meclizine/Kollidon VA 64/formic acid at a ratio of 1:2:I onto 25-30 mesh sugar spheres.

There was a need for a method for preparing spray dried solid dispersion of an active agent, AA, which is a weak organic base in its free base form, and dispersion polymers, which allows for dissolving the APIs in easily processable spray drying solvents such as C₁₋₃ alkanols at modest temperature, i.e. a temperature below the ambient pressure boiling point, at sufficiently high concentrations to enable economical throughput of SDDs. By the method the free base should be obtained as a SDD. The amorphous solid dispersion should be stable over a longer period of time.

It was found that formic acid may be used as processing aid in such spray drying method. The solubility of the AA is increased, which allows for higher concentration of AA in the spray solution than in absence of formic acid. Increased AA solubility gives higher manufacturing throughput, and potentially better spray dried particle characteristics than what is achievable with lower solids content spray solutions. When formic acid is used not only as a processing aid, but as the only solvent then viscosities tend to be high.

### Abbreviations and Definitions Used in this Specification

AA active agent
Active agent As used herein, the term “active agent” refers to a component that exerts a desired physiological effect on a mammal, including but not limited to humans. Synonymous terms include “active ingredient,” “active substance,” “active component,” “active pharmaceutical ingredient,” and “drug”.
Amorphous Substantially non-crystalline. Amorphous solids lack a definite crystalline structure and a sharp, well-defined melting point; instead, an amorphous solid melts gradually over a range of temperatures.
API active pharmaceutical ingredient
ASD amorphous solid dispersion
dasatinib CAS 302962-49-8, basic pKa 7.19 according to https://go.drugbank.com/drugs/DB01254, MW 488 g/mol (anhydrous/crystalline)
Dispersion: A system in which particles are distributed in a continuous phase of a different composition. A solid dispersion is a system in which at least one solid component is distributed in another solid component.
DISPPOL dispersion polymer
eq molar equivalents
gefitinib crystalline gefitinib, CAS 184475-35-2, basic pKa=6.85 according to https://go.drugbank.com/drugs/DB00317, MW 446.9 g/mol
HPMCAS Hydroxypropyl Methylcellulose Acetate Succinate, Hypromellose Acetate Succinate, CAS 71138-97-1
MW molecular weight
nilotinib CAS 641571-10-0, basic pKa=5.92 according to https://go.drugbank.com/drugs/DB04868, MW 529.5 g/mol
pKa the pKa of a basic site of an organic Bronstedt base is the pH at which half of these basic sites are protonated. At a pH which is lower than this basic pKa more than half of these basic sites are protonated, that is ionized. This pKa of a basic site is also called basic pKa.
   In contrast thereof the pKa of an acidic site of an organic Bronstedt acid is the pH at which half of these acidic sites are deprotonated, that is ionized. At a pH which is higher than this acidic pKa more than half of these acidic sites are deprotonated. This pKa of an acidic site is also called acidic pKa.
   pKa values are available in the internet, they may also be calculated, for example by ADMET Predictor® software, Simulations Plus, Inc. (Nasdaq: SLP) or measured in the lab.
PVP-VA Vinylpyrrolidone-vinyl acetate copolymer
RT room temperature; for the purpose of this invention RT means temperatures from 20 to 25° C.
SDD Spray dried solid dispersions
solubilities The solubilities stated herein in mg/mL or mg/g are mg of the respective substance per mL or per mg solvent,
   solubilities stated herein in wt % are weight of dissolved substance per weight of solvent;
   any solubilities herein are determined at room temperature as defined herein, a typical value is 25° C.;
   if not stated explicitly otherwise
Solubilize To make soluble or increase the solubility of.
Solution A homogeneous mixture composed of two or more substances. A solute (minor component) is dissolved in a solvent (major component). In contrast to a suspension, light passes through a solution without scattering from solute particles.
SPRAYDRY method for preparing a spray dried solid dispersion SDD
SPRAYSOL spray solution. As used herein, the term “spray solution” refers to a fluid formed by dissolving an active agent and a dispersion polymer in a solvent and an amount of ammonia. In the case of the active agent, the term “dissolved” has the conventional meaning, indicating that the active agent has gone into solution when combined with the solvent and the amount of ammonia. In the case of dispersion polymers, the term “dissolved” can take a broader definition. For some dispersion polymers, the term dissolved can mean that the dispersion polymer has gone into solution and has dissolved in the conventional sense, or it can mean that the dispersion polymer is dispersed or highly swollen with the solvent such that it acts as if it were in solution, or it can mean that a portion of the dispersion polymer molecules are in solution and the remaining dispersion polymer molecules are dispersed or highly swollen with solvent. Any suitable technique may be used to determine if the active agent and dispersion polymer are dissolved. Examples include dynamic or static light scattering analysis, turbidity analysis, and visual observations.
wt % weight %

### SUMMARY OF THE INVENTION

Subject of the invention is a method SPRAYDRY for preparing a spray dried solid dispersion, SDD, of an active agent, AA, which is an organic Bronstedt base, comprising:
a. combining an active agent, AA, a dispersion polymer, DISPPOL, formic acid, and a solvent, SOLV, to form a spray solution, SPRAYSOL, wherein
   i. SOLV comprises a C₁₋₃ alkanol, the amount of the C₁₋₃ alkanol in SOLV is at least 50 wt %, with the wt % being based on the weight of SOLV;
   ii. AA is in its free base form when combined with the formic acid and SOLV to form SPRAYSOL, and, in its free base form, has a basic pKa of 3 or greater, and AA has a solubility at RT of 40 mg/mL or less in SOLV,
   iii. SPRAYSOL is not a supersaturated solution of AA in SOLV and formic acid;
b. spray drying SPRAYSOL to form a SDD comprising AA and DISPPOL; SPRAYSOL has only one liquid phase.

### DESCRIPTION OF THE DRAWINGS

FIG. 1: PXRD diffractogram showing the amorphous nature of the SDD, which represents a gefitinib dispersion in HPMCAS-MG.

### DETAILED DESCRIPTION OF THE INVENTION

SDD is a spray dried solid dispersion of AA in DISPPOL. AA and DISPPOL are preferably homogeneously mixed in SDD.

In a solid dispersion of AA in DISPPOL, AA may be homogeneously and preferably also molecularly dispersed in DISPPOL. AA and DISPPOL may form a solid solution in SDD.

AA may be amorphous or substantially amorphous in SDD; substantially means that at least 80 wt %, preferably at least 90 wt %, more preferably at least 95 wt %, even more preferably at least 98 wt %, especially at least 99% wt %, of AA is amorphous; the wt % being based on the total weight of AA in SDD. SDD therefore may be an amorphous SDD. The amorphous nature of AA may be evidenced by a lack of sharp Bragg diffraction peaks in the x-ray pattern when SDD is analyzed by a powder X-Ray Diffraction, PXRD. Possible parameters and settings for a x-ray diffractometer are equipment with a Cu-Kalpha source, setting in modified parallel beam geometry between 3 and 40° 2Theta and a scan rate of 2°/min with a 0.0° step size. Another evidence for the amorphous nature of AA in the SDD may be a single glass transition temperature, Tg. A single Tg is also evidence of a homogeneous mixture of amorphous AA and polymer. Samples as such without any further sample preparation may be used for the determination of the Tg, the determination may run for example in modulated mode at a scan rate of 2.5° C./min, modulation of +1.5° C./min, and a scan range from 0 to 180° C. Amorphous nature of AA shows a Tg which is equal to the Tg of neat DSISPPOL or which is between the Tg of the polymer and the Tg of the AA. The Tg of the SDD is often similar to the weighted average of the Tg of AA and the Tg of DISPPOL. SDD is amorphous or substantially, SDD can also be called amorphous solid dispersion, ASD.

SPRAYSOL is a stable solution of AA in SOLV and formic acid.

SPRAYSOL has only one liquid phase.

SPRAYSOL does not contain AA in solid form.

Preferably, SPRAYSOL does not contain HCl.

Preferably, SPRAYSOL does not contain citric acid.

More preferably, SPRAYSOL does not contain HCl and does not contain citric acid.

Even more preferably, SPRAYSOL does not contain any other acid besides the formic acid.

Especially, SPRAYSOL consists of AA, DISPPOL, formic acid and SOLV, with AA, DISPPOL, formic acid and SOLV as defined herein, also with their embodiments.

The amount of AA with respect to SOLV is above the solubility of AA in SOLV in absence of formic acid.

Possible amount of AA in SPRAYSOL may be at least 0.5 wt %, preferably at least 1 wt %, more preferably at least 3 wt %, with the wt % being based on the weight of SPRAYSOL.

Possible amount of AA may be up to 10 wt %, preferably up to 7.5 wt %, more preferably up to 5 wt %.

Any of the lower limits may be combined with any of the upper limits of AA in SPRAYSOL.

For example, possible amounts of AA in SPRAYSOL may be from 0.5 wt % to 10 wt %, preferably from 1 wt % to 10 wt %, more preferably from 2 wt % to 10 wt %, with the wt % being based on the weight of SPRAYSOL.

The amount of formic acid is sufficient to solubilize AA in SOLV.

The amount of formic acid may be from 1 to 50 eq, preferably from 1 to 40 eq, more preferably from 1 to 30 eq, even more preferably from 1 to 25 eq, especially from 1 to 20 eq, more especially from 1 to 15 eq, even more especially from 1 to 10 eq, in particular from 1 to 5 eq, more in particular from 2 to 5 eq, even more in particular from 3 to 5 eq, based on the molar amount of AA.

In a particular embodiment, the amount of formic acid is from 3.5 to 4.5 eq based on the molar amount of AA, with 4 eq being a possible specific value.

In another particular embodiment, the amount of formic acid is from 3 to 11 eq based on the molar amount of AA.

The amount of formic acid may be up to 50 wt %, preferably up to 40 wt %, more preferably up to 30 wt %, even more preferably up to 25 wt %, especially up to 15 wt %, more especially up to 10 wt %, even more especially up to 7.5, in particular up to 5 wt %, more in particular up to 2 wt %, the wt % being based on the weight of SOLV.

The amount of formic acid may be from 0.05 to 50 wt %, preferably from 0.05 to 40 wt %, more preferably from 0.05 to 30 wt %, even more preferably from 0.05 to 25 wt %, especially from 0.05 to 15 wt %, more especially from 0.1 to 10 wt %, even more especially from 0.1 to 7.5, in particular from 0.1 to 5 wt %, more in particular from 0.1 to 2 wt %, even more in particular from 0.3 to 2 wt %, the wt % being based on the weight of SOLV.

The C₁₋₃ alkanol of SOLV may be methanol, ethanol or isopropanol, preferably methanol or ethanol, more preferably methanol.

The amount of the C₁₋₃ alkanol in SOLV may be at least 60 wt %, or at least 65 wt %, or at least 67.5 wt %, or at least 70 wt %, or at least 75 wt %, or at least 80 wt %, or at least 85 wt %, or at least 90 wt %, or at least 95 wt %; with the wt % being based on the weight of SOLV.

In another embodiment, SOLV may further comprise water.

When SOLV comprises water, then SOLV comprises not more than 40 wt %, preferably not more than 35 wt %, more preferably not more than 32.5 wt %, even more preferably not more than 30 wt %, especially not more than 27.5 wt %, more especially not more than 25 wt %, even more especially not more than 22.5 wt %, in particular not more than 20 wt %, of water;
also lower amounts of water may be used, such as not more than 15 wt %, or not more than 10 wt %, or not more than 5 wt %, of water;
with the wt % being based on the combined weights of C₁₋₃ alkanol and water, in another embodiment with the wt % being based on the weight of SOLV.

When the SOLV comprises water, then SOLV may comprise at least 0.5 wt %, preferably at least 1 wt %, more preferably at least 2 wt %; even more preferably at least 5 wt %, of water, with the wt % being based on the combined weights of C₁₋₃ alkanol and water, in another embodiment with the wt % being based on the weight of SOLV.

When SOLV comprises water, then the weight ratio C₁₋₃ alkanol:water in SOLV may be from 99:1 to 60:40, preferably from 99:1 to 65:35, more preferably from 99:1 to 67.5:32.5, even more preferably from 99:1 to 70:30, especially from 99:1 to 75:25, more especially from 99:1 to 80:20, even more especially from 95:5 to 80:20, in particular from 90:10 to 80:20;
another possible weight ratio may be from 95:5 to 75:25.

When SOLV comprises water, then the combined amount of the C₁₋₃ alkanol and water in SOLV may be at least 60 wt %, or at least 65 wt %, or at least 67.5 wt %, or at least 70 wt %, or at least 75 wt %, or at least 80 wt %, or at least 85 wt %, or at least 90 wt %, or at least 95 wt %; with the wt % being based on the weight of SOLV.

In one embodiment, SOLV consists of C₁₋₃ alkanol and water; preferably, the C₁₋₃ alkanol is MeOH.

In one embodiment, SOLV consists of C₁₋₃ alkanol; preferably, the C₁₋₃ alkanol is MeOH.

In a possible particular embodiment SOLV may be consist of C₁₋₃ alkanol and water with a weight ratio C₁₋₃ alkanol:water from 95:5 to 75:25;
preferably, the amount of formic acid is from 1 to 5 eq, more preferably from 2 to 5 eq, even more preferably from 3 to 5 eq, especially 3.5 to 4.5 eq, based on the molar amount of AA.

In another possible particular embodiment SOLV may be consist of MeOH and water with a weight ratio C₁₋₃ alkanol:water from 95:5 to 75:25;
preferably, the amount of formic acid is from 1 to 5 eq, more preferably from 2 to 5 eq, even more preferably from 3 to 5 eq, especially 3.5 to 4.5 eq, based on the molar amount of AA.

AA is in its free base form when combined with the formic acid and SOLV to form SPRAYSOL.

AA may be present in SPRAYSOL in its free base form or in its protonated form.

The SDD may comprise from 1 to 99 wt %, preferably from 10 to 95 wt %, more preferably from 10 to 80 wt %, even more preferably from 20 to 60 wt %, especially from 20 to 40 wt %, more especially from 20 to 30 wt %, of AA, the wt % being based on the weight of the SDD.

The SDD may comprise from 1 to 99 wt %, preferably from 20 to 90 wt %, more preferably from 40 to 80 wt %, even more preferably from 60 to 80 wt %, especially from 70 to 80 wt %, of DISPPOL, the wt % being based on the weight of the SDD.

Preferably, the combined content of AA and DISPPOL in SDD is from 65 to 100 wt %, more preferably from 67.5 to 100 wt %, even more preferably from 80 to 100 wt %; especially from 90 to 100 wt %; more especially from 95 to 100 wt %;
the wt % being based on the weight of the SDD;
in one embodiment, the SDD consists of AA and DISPPOL.

Relative amounts (w/w) of AA to DISPPOL in SDD may be from 50:1 to 1:50, preferably from 25:1 to 1:25, more preferably from 10:1 to 1:10, even more preferably from 1:1 to 1:10, especially from 1:1 to 1:5.

Amounts of DISPPOL and of AA in SPRAYSOL are chosen such that a predefined amount of DISPPOL and of AA in SDD provided.

DISPPOL is present in SPRAYSOL in a dissolved state, the amounts of DISPPOL and SOLV are chosen respectively.

For example amounts of DISPPOL in SPRAYSOL may be from 0.5 wt % to 25 wt %, preferably from 1 wt % to 20 wt %, more preferably from 2.5 wt % to 15 wt %, even more preferably from 3 wt % to 10 wt %, especially from 5 wt % to 10 wt %, with the wt % being based on the weight of SPRAYSOL.

Preferably, AA may have a solubility of 30 mg/mL or less, more preferably of 20 mg/mL or less, even more preferably of 10 mg/mL or less, in SOLV.

AA is an organic Bronstedt base.

AA may be a biologically active compound. The biologically active compound may be desired to be administered to a patient in need of AA.

AA may be a drug, medicament, pharmaceutical, therapeutic agent, nutraceutical, agrochemical, fertilizer, pesticide, herbicide, nutrient, or an active pharmaceutical ingredient, API; preferably an API.

AA may be a “small molecule,” generally having a molecular weight of 2000 Daltons or less.

An API, that is AA, may be dasatinib or gefitinib.

AA may be one or more AAs; SDD may contain one or more AAs.

Preferably, AA has a low solubility in SOLV, especially in methanol, e.g. a low solubility of less than 3 wt %, or even less than 2 wt %, or even less than 1 wt %, or even less than 0.5 wt %, or even less than 0.25 wt %.

Preferably the solubility of AA in a mixture of SOLV and formic acid, which has a composition of SOLV and formic acid as is present in SPRAYSOL, is at least 1.1 fold, more preferably at least 1.25 fold, even more preferably at least 1.5 fold, even more preferably at least 1.75 fold, even more preferably at least 2 fold, especially at least 3 fold, more especially at least 4 fold, higher than the solubility of AA in SOLV alone without formic acid.

Preferably the solubility of said AA in SPRAYSOL is at least 1.1 fold, more preferably at least 1.25 fold, even more preferably at least 1.5 fold, even more preferably at least 1.75 fold, even more preferably at least 2 fold, especially at least 3 fold, more especially at least 4 fold, higher than the solubility of AA in SOLV alone without formic acid.

Preferably the concentration of AA dissolved in a mixture of SOLV and formic acid, which has a composition of SOLV and formic acid as is present in SPRAYSOL, is at least 1.1 fold, more preferably at least 1.25 fold, even more preferably at least 1.5 fold, even more preferably at least 1.75 fold, even more preferably at least 2 fold, especially at least 3 fold, more especially at least 4 fold, higher than the solubility of AA in SOLV alone without formic acid.

Preferably the concentration of said AA dissolved in SPRAYSOL is at least 1.1 fold, more preferably at least 1.25 fold, even more preferably at least 1.5 fold, even more preferably at least 1.75 fold, even more preferably at least 2 fold, especially at least 3 fold, more especially at least 4 fold, higher than the solubility of AA in SOLV alone without formic acid.

In SPRAYDRY both SOLV and the formic acid are evaporated.

SPRAYDRY recovers and provides AA in its free base form, so essentially all of AA is obtained and is present in the SDD in its free base form, this means that the basic site of AA which has said basic pKa of 3 or greater is essentially present in deprotonated state in the SDD.

Preferably, AA has a basic pKa of 4 or greater, more preferably of 5 or greater, even more preferably of 6 or greater.

DISPPOL may comprise one or more dispersion polymers, preferably 1, 2, 3 or 4, more preferably 1, 2 or 3, even more preferably 1 or 2 dispersion polymers.

DISPPOL may be a pharmaceutically acceptable dispersion polymer.

Suitable DISPPOL include, but are not limited to, hydroxypropyl methylcellulose acetate succinate (HPMCAS), hydroxypropyl methylcellulose phthalate (HPMCP), hydroxypropyl methyl cellulose (HPMC), hydroxypropyl cellulose (HPC), cellulose acetate phthalate (CAP), carboxymethyl ethyl cellulose (CMEC), polyvinylpyrrolidone (PVP), poly(vinylpyrrolidone-co-vinyl acetate) (PVP-VA), poly(methacrylic acid-co-methyl methacrylate) (PMMAMA), poly(methacrylic acid-co-ethyl acrylate), or any combination thereof.

Suitable PMMAMA polymers include, but are not limited to, poly(methacrylic acid-co-methyl methacrylate) 1:1 (for example Eudragit® L100), and poly(methacrylic acid-co-methyl methacrylate) 1:2 (for example Eudragit® S100). Eudragit® are polymer products of Evonik Industries AG, 45128 Essen, Germany.

The poly(methacrylic acid-co-ethyl acrylate) may be poly(methacrylic acid-co-ethyl acrylate) 1:1.

In an embodiment, DISPPOL is hydroxypropyl methyl cellulose, PVP, PVP-VA, HPMCAS or poly(methacrylic acid-co-methyl methacrylate).

In an embodiment, DISPPOL is HPMCAS or PMMAMA.

In another embodiment, DISPPOL is hydroxypropyl methyl cellulose;
in another embodiment DISPPOL is PVP or PVP-VA;
in another embodiment DISPPOL is HPMCAS.

Preferred embodiments of HPMCAS are
HPMCAS with an acetyl content from 5 to 9 wt % and a succinoyl content from 14 to 18 wt %,
HPMCAS with an acetyl content from 7 to 11 wt % and a succinoyl content from 10 to 14 wt %, or
HPMCAS with an acetyl content from 10 to 14 wt % and a succinoyl content from 4 to 8 wt %;
   more preferably
HPMCAS with an acetyl content from 5 to 7 wt % and a succinoyl content from 14 to 16 wt %,
HPMCAS with an acetyl content from 7 to 9 wt % and a succinoyl content from 10 to 12 wt %, or
HPMCAS with an acetyl content from 11 to 13 wt % and a succinoyl content from 5 to 7 wt %;
   with the wt % being based on the weight of HPMCAS.

The dispersion polymer and the mixed solvent are chosen such that the dispersion polymer dissolves in the mixed solvent.

When DISPPOL is hydroxypropyl methyl cellulose then preferably SOLV comprises water, with the amount of water and all its embodiments as stated herein, for example from 10 to 30 wt %, or from 15 to 30 wt %, or from 20 to 30 wt %, with the wt % being based on the weight of SOLV.

The combining of AA, DISPPOL, formic acid, and SOLV, to form SPRAYSOL may be done in any sequence, such as in a first step combining formic acid with SOLV for provide a mixture of formic acid with SOLV, thereafter in a second step adding AA to said mixture to provide a solution of AA in said mixture, thereafter in a third step adding DISPPOL to said solution; or
adding DISPPOL in said second step and AA in said third step.

SPRAYSOL may be fed into the spray dryer with a temperature of SPRAYSOL up to the boiling point of SPRAYSOL at ambient pressure; preferably with a temperature of from 4° C. to the boiling point of SPRAYSOL at ambient pressure, preferably from 4° C. to a temperature below the boiling point of SPRAYSOL at ambient pressure, more preferably from room temperature to 60° C. In the context of this invention the term “SPRAYSOL may be fed into the spray dryer with a temperature of SPRAYSOL” means that “SPRAYSOL is spray dried with a temperature of SPRAYSOL”.

The spray drying may be done with an inlet temperature of from 60 to 165° C., preferably from 80 to 140° C.

The spray drying may be done with an outlet temperature equal to or less than the boiling point of SOLV, such as with an outlet temperature from 20° C. to a temperature of 10° C. below the boiling point of SOLV.

The spray drying may be done with any inert gas commonly used for spray drying, such as nitrogen.

SPRAYSOL may further comprises a dissolved surfactant SURF.

SURF may be mixed with SPRAYSOL.

SURF may be for example a fatty acid and alkyl sulfonate, docusate sodium (for example available from Mallinckrodt Spec. Chern., St. Louis, Mo.), polyoxyethylene sorbitan fatty acid esters (for example Tween®, available from ICI Americas Inc, Wilmington, Del., or Liposorb® P-20, available from Lipochem Inc, Patterson, N.J., or Capmul® POE-0, available from Abitec Corp., Janesville, Wis.), natural surfactants such as sodium taurocholic acid, 1-palmitoyl-2-oleoyl-sn-glycero-3-phosphocholine, lecithin, other phospholipids and mono- and diglycerides, vitamin E TPGS, PEO-PPO-PEO triblock copolymers (for example known under the tradename pluronics), or PEO (PEO are also called PEG, polyethyleneglycols (PEG)).

The amount of SURF may be up to 10 wt %, the wt % being based on the weight of SDD.

SPRAYSOL may further comprises pharmaceutically acceptable excipients, such as fillers, disintegrating agents, pigments, binders, lubricants, flavorants, and so forth which can be used for customary purposes and in typical amounts known to the person skilled on the art.

The SDD may comprise residual formic acid, preferably in low amounts; the content of residual formic acid in SDD may be 5'000 ppm or less, preferably 500 ppm or less, more preferably of 100 ppm or less, the ppm being based on the weight of SDD.

Also after the spraying any content of residual formic acid in SDD may be lowered to a predefined content of residual formic acid, this may be done with an additional drying step after spray drying.

The SDD may comprise residual SOLV, the content of residual SOLV in SDD may be 5'000 ppm or less, preferably 3'000 ppm or less, more preferably 500 ppm or less, even more preferably of 100 ppm or less, the ppm being based on the weight of SDD.

Also after the spraying any content of residual SOLV in SDD may be lowered to a predefined content of residual SOLV in SD.

Any residual content of formic acid or of SOLV in SDD may be reduced to the desired predefined and final content by submitting SDD after the spray drying to a second drying. Secondary drying may be done using a tray dryer or any agitated dryer known to the skilled person for drying solids.

Further subject of the invention is a spray dried solid dispersion, SDD, wherein the SDD is obtainable by the method SPRAYDRY;
with SDD and SPRAYDRY as defined herein, also with all their embodiments.

### Examples

### Materials and Abbreviations

dasatinib crystalline Dasatinib free base, LC Laboratories, Woburn, MA, USA, >99+%
formic acid 98%, EMD Millipore Corporation, an affiliate of Merck KGaA, Darmstadt, Germany
gefitinib gefitinib free base, LC Laboratories, Woburn, MA, USA
GC gas chromatography
HPMCAS-MG AquaSolve, Ashland, pharma grade
HPMC E3 Methocel E3, Hydroxypropyl Methylcellulose, Manufacturer DuPont de Nemours, Inc., Wilmington, Delaware, USA
nilotinib free base, LC Laboratories, Woburn, MA, USA
PMMAMA Eudragit L-100, Evonik, pharma grade.
PVP-VA64 PVP-VA, Kollidon VA64 from BASF, Ludwigshafen, Germany
PXRD powder x-ray diffraction
RCF relative centrifugal force
RH relative humidity
TGA thermo gravimetric analysis

### Example 1: Solubility of Dasatinib in Methanol

Dasatinib free base was recrystallized from methanol and dried. Crystalline dasatinib was added in excess to methanol to form a saturated solution at 25° C. The solution was analyzed by TGA and found to contain 3.1 mg/mL dasatinib.

### Example 2: Increasing Dasatinib Concentration Dissolved in Methanol with Formic Acid

200 mg of crystalline dasatinib was slurried in 10 mL of methanol at 24° C. Upon adding 100 microliter (6.4 eq based on the molar amount of dasatinib) of 98% formic acid the mixture became a clear solution. The concentration of dissolved dasatinib was approximately 19.8 mg/mL and ca. 6 fold higher than the solubility in methanol without formic acid.

### Example 3: Gefitinib Solubilities

Crystalline gefitinib was added in excess to methanol and methanol:water mixtures to form saturated solutions at 20° C. After 24 hour of stirring, 1 mL aliquots were centrifuged at 10'000 RCF for 3 min. The supernatant was then analyzed for gefitinib concentration by HPLC. Solubilities in MeOH:water mixtures are given in Table 1.

### Example 4: Gefitinib Solubilities with Formic Acid

The solubility of gefitinib in solvent mixtures with 98% formic acid were obtained by suspending at RT 300 mg of crystalline gefitinib in 5 mL of solvent containing 100 microliters of formic acid at 21° C. (4.0 eq of formic acid). Both solutions were visually clear solutions at 60 mg/mL.

5 ml of a mixture of 4.9 ml of 90:10 methanol/water w/w with 0.1 ml HCOOH weigh 4.122 g based on the densities at RT as stated below.

5 ml of a mixture of 4.9 ml 80:20 methanol:water w/w with 0.1 ml HCOOH weigh 4.212 g based on the densities at RT as stated below.

The solution in the mixture of 4.9 ml of 90:10 MeOH:H₂O w/w with 0.1 ml HCOOH was a visually clear solution at 72.8 mg/g based on the densities at RT as stated below.

The solution in the mixture of 4.9 ml of 80:20 MeOH:H₂O w/w with 0.1 ml HCOOH was a visually clear solution at 71.2 mg/g based on the densities at RT as stated below.

Table 1 shows the gefitinib solubility enhancement in solvent mixtures using 4.0 eq of formic acid; the solubility enhancement is expressed in Table 1 in form of an Solubility Enhancement Factor which is the ratio of

[Solubility with formic acid]/[Solubility without formic acid]

The solubilities in Table 1 in mg/mL or mg/g are mg gefitinib per mL or per mg solvent, if not explicitly stated otherwise.

TABLE 1

| | | | | | Solubility | |
|---|---|---|---|---|---|---|
| Solvent | Solubility | | Solubility | | Enhancement | |
| composition | without | | with formic | | factor | |
| MeOH:H₂O | formic acid | [mg/g] | acid (*) | [mg/g] | based on | based on |
| (w/w) | [mg/mL] | (**) | [mg/mL] | (**) | [mg/mL] | [mg/g] |
| 100:0 | 7.0 | 8.8 | 30 | 37.5 | 4.3 | 4.3 |
| 90:10 | 6.0 | 7.4 | >60 | >72.8 | >10 | >9.9 |
| 80:20 | 6.3 | 7.6 | >60 | >71.2 | >9.5 | >9.3 |

(*) 4.0 molar equivalents of formic acid added
(**) calculated based on densities at RT:
MeOH 0.8 g/ml (assumed)
water 1 g/ml (assumed)
MeOH:water 90:10 w/w 0.816 g/ml (calculated)
MeOH:water 80:20 w/w 0.833 g/ml (calculated)
Mixture of 4.9 ml MeOH:H₂O 90:10 w/w with 0.1 ml HCOOH 0.8244 g/ml (calculated)
Mixture of 4.9 ml MeOH:H₂O 80:20 w/w with 0.1 ml HCOOH 0.8424 g/ml (calculated)
HCOOH 1.22 g/ml (assumed) MW 46 mg/mmol

### Example 5: Nilotinib Solubilities with Formic Acid

In analogy to Example 4 the solubilities of nilotinib at 25° C. were determined.

Table 2 shows the respective nilotinib solubility enhancement in solvent mixtures using 4.0 eq of formic acid; the solubility enhancement is expressed in Table 2 in form of an Solubility Enhancement Factor which is the ratio of

$$[ Solubility with formic acid ] / [ Solubility without formic acid ]$$

The solubilities in Table 1 in mg/mL are mg nilotinib per mL solvent, if not explicitly stated otherwise.

TABLE 2

| | | | Solubility |
|---|---|---|---|
| Solvent | Solubility | Solubility | Enhancement |
| composition | without | with | factor |
| MeOH:H₂O | formic acid | formic acid(*) | based on |
| (w/w) | [mg/mL] | [mg/mL] | [mg/mL] |
| 100:0 | 2.9 | 7.9 | 2.7 |
| 90:10 | 2.0 | 8.2 | 4.1 |
| 80:20 | 1.0 | 8.2 | 8.2 |

(*)4.0 molar equivalents of formic acid added

Addition of formic acid with and without the presence of water results in an increase of solubility of nilotinib over the solubility in MeOH.

### Example 6: SDD Using Formic Acid as Processing Aid with Methanol—25/75 Gefitinib:HPMCAS-MG

37.6 g of methanol as SOLV, 4.4 g water, and 0.4 g formic acid (8.696 mmol, 3.9 eq) were added to a flask to make a solvent blend 88.7/10.4/0.9 (w/w) methanol/water/formic acid.

1.0088 g (2.257 mmol) of gefitinib was added to the flask and stirred with a magnetic stir bar at room temperature (20° C.). Once the drug dissolved and the solution was clear, 3.0086 g of HPMCAS-MG was added to the same flask and stirred until dissolved providing a spray solution SPRAYSOL with 2.2 wt % of gefitinib and 6.5 wt % of HPMCAS-MG based on the weight of SPRAYSOL.

SPRAYSOL did not contain gefitinib in solid form, instead it contained the gefitinib in a completely dissolved state, and it had only one liquid phase.

The calculated volume of the solvent was 47 ml+4.4 ml+0.33 ml=51.73 ml based on the assumed densities stated in Example 4.

The calculated concentration of gefitinib was:
19.5 mg/ml (based on volume of solvent)
23.7 mg/g (based on weight of solvent)

The solution was spray dried using a custom built spray dryer. The solution was pumped into a lab-scale 0.3 m diameter stainless steel spray drying chamber using a peristaltic pump to feed the solution to the nozzle at a flowrate of 15 g/min. A two-fluid nozzle ¼ J series with a 1650 liquid body and a 64 air cap made by Spraying Systems Company, Glendale Heights, IL 60187-7901, United States. Nitrogen gas as sheath gas was used to atomize the solution at a pressure of 20 psi. Heated nitrogen gas (140° C. inlet, 50 to 52° C. outlet, 500 g/min) was used to dry the particles. The resulting SDD was collected using a cyclone to separate the solids from the gas stream.

The collected SDD was placed in a vacuum tray dryer at 40° C. for secondary drying with 3.5 slpm nitrogen sweep for 24 hours at a pressure of 0.2 atm.

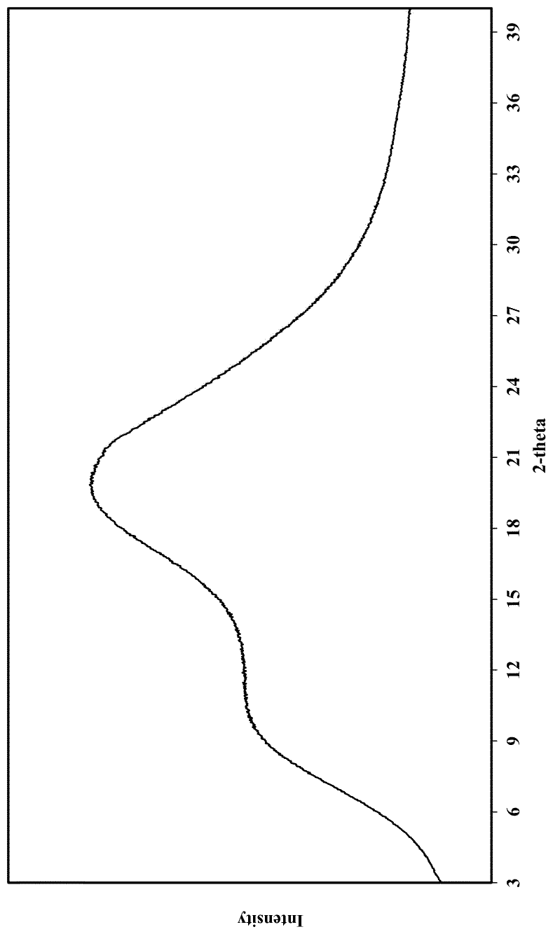

FIG. 1 shows the PXRD of the collected SDD, which represents a gefitinib dispersion in HPMCAS-MG, and it confirms the amorphous nature of the SDD.

1. A method for preparing a spray dried solid dispersion of an active agent, comprising:
a. combining an active agent, which is an organic Bronsted base, a dispersion polymer, formic acid, and a solvent, to form a spray solution, wherein
i. the solvent comprises a C₁₋₃ alkanol,
an amount of the C₁₋₃ alkanol in the solvent is at least 50 wt %, with the wt % being based on the weight of the solvent,
ii. the active agent is in its free base form when combined with the formic acid and the solvent to form the spray solution, and, in its free base form, has a basic pKa of 3 or greater, and the active agent has a solubility of 40 mg/ml or less at room temperature in the solvent, and
iii. the spray solution is not a supersaturated solution of the active agent in the solvent and formic acid; and
b. spray drying the spray solution to form a spray dried solid dispersion comprising the active agent and the dispersion polymer;
wherein the spray solution has only one liquid phase, and
the active agent is a drug, a medicament, a pharmaceutical, a therapeutic agent, a nutraceutical, or an active pharmaceutical ingredient.

2. The method according to claim 1, wherein the active agent is an active pharmaceutical ingredient.

3. The method according to claim 1, wherein an amount of the active agent with respect to the solvent is above the solubility of the active agent in the solvent in absence of the formic acid.

4. The method according to claim 1, wherein an amount of the active agent in the spray solution is at least 0.5 wt %, with the wt % being based on the weight of the spray solution.

5. The method according to claim 1, wherein an amount of the formic acid is 1 eq to 50 eq based on a molar amount of the active agent.

6. The method according to claim 1, wherein an amount of the formic acid is from 0.05 wt % to 50 wt %, the wt % being based on the weight of the solvent.

7. The method according to claim 1, wherein the solvent is methanol, ethanol, or isopropanol.

8. The method according to claim 1, wherein the solvent further comprises water.

9. The method according to claim 8, wherein a weight ratio of C₁₋₃ alkanol:water in the solvent is from 99:1 to 60:40.

10. The method according to claim 1, wherein the spray dried solid dispersion comprises from 1 wt % to 99 wt % of the active agent, the wt % being based on the weight of the spray dried solid dispersion.

11. The method according to claim 1, wherein the spray dried solid dispersion comprises from 1 wt % to 99 wt % of the dispersion polymer, the wt % being based on the weight of the spray dried solid dispersion.

12. The method according to claim 1, wherein a combined content of the active agent and the dispersion polymer in the spray dried solid dispersion is from 65 wt % to 100 wt %, the wt % being based on the weight of the spray dried solid dispersion.

13. The method according to claim 1, wherein the active agent is a biologically active compound.

14. The method according to claim 1, wherein the active agent has a basic pKa of 4 or greater.

15. The method according to claim 1, wherein the dispersion polymer comprises a plurality of dispersion polymers.

16. The method according to claim 1, wherein the dispersion polymer is a pharmaceutically acceptable dispersion polymer.

17. The method according to claim 1, wherein the dispersion polymer is hydroxypropyl methylcellulose acetate succinate (HPMCAS), hydroxypropyl methylcellulose phthalate, hydroxypropyl methyl cellulose (HPMC), hydroxypropyl cellulose, cellulose acetate phthalate, carboxymethyl ethyl cellulose, polyvinylpyrrolidone (PVP), poly(vinylpyrrolidone-co-vinyl acetate) (PVP-VA), poly(methacrylic acid-co-methyl methacrylate) (PMMAMA), poly(methacrylic acid-co-ethyl acrylate), or any combination thereof.

18. The method according to claim 1, wherein the dispersion polymer is HPMC, PVP, PVP-VA, HPMCAS, or PMMAMA.

19. The method according to claim 1, wherein;
the spray dried solid dispersion comprises residual formic acid; and
a content of residual formic acid in the spray dried solid dispersion is 5000 ppm or less, the ppm being based on the weight of the spray dried solid dispersion.

20. The method according to claim 1, wherein the solvent consists of C₁₋₃ alkanol.

21. The method according to claim 1, wherein the solvent consists of C₁₋₃ alkanol and water.
